SEQUENCE LISTING (4 sequences)

SEQ ID NO: 1 - DNA, 26 bp, artificial sequence
  primer
gaaagtcgaggagacagttaagcatg

SEQ ID NO: 2 - DNA, 26 bp, artificial sequence
  primer
cacaaggaaaggtcatgctaaaactg

SEQ ID NO: 3 - RNA, 21 nt, artificial sequence
  test siRNA
ccauagagggcgaauuuaauu

SEQ ID NO: 4 - RNA, 21 nt, artificial sequence
  scramble siRNA
gcuccuuucgucucacauauu

(19) United States
(12) Patent Application Publication
(10) Pub. No.: US 2015/0050682 A1
(43) Pub. Date: Feb. 19, 2015

(54) DIRECT ASSAY OF THIOREDOXIN REDUCTASE ACTIVITY

(71) Applicant: University of Vermont and State Agricultural College, Burlington, VT (US)

(72) Inventors: Robert J. Hondal, Burlington, VT (US); Nicholas H. Heintz, Jericho, VT (US); Brian Cunniff, Burlington, VT (US); Nicholas Fredette, Essex, VT (US); Gregg W. Snider, Atlanta, GA (US)

(21) Appl. No.: 14/460,759

(22) Filed: Aug. 15, 2014

Related U.S. Application Data
(60) Provisional application No. 61/866,165, filed on Aug. 15, 2013.

(51) Int. Cl. C12Q 1/26

(52) U.S. Cl. CPC C12Q 1/26; G01N 2333/90212
USPC 435/25

(57) ABSTRACT

Provided is a direct method for detecting thioredoxin reductase (TR) activity in test samples. The method can provide a continuous and real-time measurement of TR activity. The method comprises contacting the test sample with NADPH and a diselenide substrate of TR, and then measuring conversion of NADPH to NADP. Also provided are kits for use in the method of direct detection of TR activity.

### CROSS-REFERENCE TO RELATED APPLICATIONS:

This application claims priority to U.S. Provisional application No. 61/866,165, filed on Aug. 15, 2013, the disclosure of which is incorporated by reference in its entirety.

### STATEMENT REGARDING FEDERAL FUNDING

This invention was made with government support under grant number GM094172 from the National Institutes of General Medical Science. The government has certain rights in the invention.

### BACKGROUND OF THE DISCLOSURE

The mammalian thioredoxin reductase system is composed of thioredoxin reductase (TR), thioredoxin (Trx), and NADPH. The thioredoxin system, along with the glutaredoxin system, function as the major antioxidant systems in the cell. Mammalian TR is a homodimeric, pyridine nucleotide oxidoreductase that contains the rare amino acid selenocysteine (Sec, U) on the C-terminal tail of the enzyme. The molecular weight of each monomer of the head-to-tail dimer is approximately 55 kDa and each subunit contains a FAD prosthetic group and utilizes NADPH as the source of hydride. Trx is the carrier of reducing equivalents to multiple protein targets within the cell including ribonucleotide reductase, methionine sulfoxide reductase, protein disulfide isomerase, peroxiredoxins, and multiple transcription factors.

Both TR and Trx are up regulated in diseases such as cancer, viral infection, and inflammation. As such, the detection of TR and Trx in biological tissues and fluids can be used as a marker of disease and oxidative stress. The presence of TR and Trx can be detected by either immunoblot analysis or from the activity of TR in cell lysates. Detection by immunoblotting is both expensive and time-consuming and does not quantify enzyme activity. The current detection method of TR activity is accomplished by an indirect, coupled assay that relies on the reduction of the disulfide bonds of insulin added to cell lysates by Trx; Trx is in turn reduced by TR, which uses NADPH as the ultimate source of electrons for the reduction. The newly reduced thiol groups of insulin are then free to react with 5,5′-dithio-bis(2-nitrobenzoic acid) (DTNB) producing a visible, yellow color that can be detected at 412 nm by spectrophotometry. This assay was originally developed for cell lysates as an end-point assay that measured the number of free thiol groups formed by the reduction of insulin (Amér et al., Methods Enzymol. 300 (1999) 226-239, Amer et al., Curr. Protoc. Toxicol. Unit 7.4 (2001)). Because it is an end-point assay it can be described as a discontinuous assay as it does not continuously monitor the consumption of NADPH by the thioredoxin system. In this discontinuous mode, the reaction is quenched with a chaotrope such as guanidinium hydrochloride and background activity comes from exposed protein thiol groups that react with DTNB. This assay has been adapted as an indirect continuous assay by commercial sources that makes use of the DTNB-reductase activity of TR (See www.sigmaaldrich.com/etc/medialib/docs/Sigma/Bulletin/1/cs0170bul.Par.0001.File.tmp/cs0170bul.pdf). One disadvantage of the commercial, continuous assay is that the cell contains other NADPH oxidoreductases that are capable of reducing DTNB such as lipoamide dehydrogenase and glutathione reductase that contribute to relatively high background activity of 15-40% (See www.sigmaaldrich.com/etc/medialib/docs/Sigma/Bulletin/1/cs0170bul.Par.0001.File.tmp/cs0170bul.pdf).

### SUMMARY OF THE DISCLOSURE

In this disclosure, we provide a direct and specific assay of TR using a water soluble diselenide containing substrate of TR and evaluating the conversion of NADPH to NADP. In one embodiment, the assay is based on the reduction of selenocystine (FIG. 1). Selenocystine is a small, commercially available diselenide-containing amino acid that is known to be only reduced by TR. The present assay can be performed by either standard spectrophotometry or may be adapted for use in other formats, including, for example, multi-well plate formats. An advantage of the present assay is that selenocystine-reductase activity of TR can be measured in the presence of non-ionic detergents (such as NP-40), which are commonly used in a wide variety of buffers to lyse mammalian cells and which inhibit activity in the previous insulin end-point assay. The present disclosure demonstrates the utility of the assay as well as the specificity of the reduction of selenocystine by TR by the use of siRNA knockdown, TR overexpression, and inhibition by acrolein, a highly specific selenol-modifying reagent.

In one embodiment, the present disclosure provides a method of detecting thioredoxin reductase activity in a biological sample comprising combining the sample with NADPH and a diselenide substrate of TR (such as selenocystine), and then measuring absorbance at 340 nm. The absorbance can be compared to a reference and an increase over the reference is indicative of TR activity in the test sample as compared to the reference. The reference may be the test sample at the start of the assay (or some other arbitrarily selected point), a positive control sample having TR activity, a negative control sample known to not have TR activity (such as from a sample where TR activity has been knocked down or blocked), or a negative control where one or more components of the assay have been deleted. In one embodiment, the TR activity is measured over a period of time to provide a real-time and continuous measure.

In another embodiment, the disclosure provides a kit for measurement of TR activity. The kit comprises NADPH, a diselenide substrate of TR (such as selenocystine), instructions for carrying out the assay, and optionally buffers.

### BRIEF DESCRIPTION OF THE DRAWINGS

FIG. 1: Reduction of selenocystine catalyzed by TR. The ultimate source of reducing equivalents comes from NADPH. The reaction can be monitored by the decrease in absorbance at 340 nm.

FIG. 2: Comparison of the end-point TR assay to SC-TR assay. A) TR activity in C10 whole cell lysates measured with the discontinuous end-point TR assay. Acrolein completely inhibits TR activity. B) TR activity in lysates from (A) measured using the SC-TR assay by monitoring the conversion of NADPH to NADP⁺ (A₃₄₀) over time in a spectrophotometer. The activity is 0.35 nmol/min or 11.7 U/mg. Acrolein completely inhibits TR activity. C and D) Overexpression of TR1 and TR2 in C10 cells increases TR activity as measured by both the end-point (C) and SC-TR assay (D). The activity of the control reaction in (D) is 0.13 nmol/min or 4.4 U/mg, while the activity of the over expressed enzyme is 0.27 nmol/min or 8.9 U/mg.

FIG. 3: Specificity of SC-TR assay to TR. A) TR activity measured using the SC-TR assay in which TR1 protein expression has been reduced using si-RNA or increased with a TR1 mammalian expression vector. B-D) SC-TR assay with individual component controls, Protein only (B), master mix only (C), and NADPH only (D). E) Western blot of C10 cells transiently transfected with si-RNA against TR1 (si-TR1) or non-specific scrambled si-RNA (scram). (F) C10 cells transfected with pcDNA 3.1 control vector or increasing concentrations of TR1 expression plasmid. Actin is used as a loading control for E and F.

FIG. 4: Versatility of SC-TR assay with lysis buffer containing NP-40. A-B) TR activity of control or auranofin (Aur) treated C10 cell lysates prepared in TE buffer (A) or NP-40 lysis buffer (B). SC-TR assay in the presence of selenocystine (left panel) or without selenocystine (right panel).

FIG. 5: TR expression and activity in human malignant mesothelioma cell and human mesothelial lysates. A) TR1 and Trx1 protein expression in LP9 mesothelial cells, HM cells, and H2373 malignant mesothelioma cells. Actin is used as a loading control. B) Densitometry of immunoblot signals normalized to actin expression (*p<0.05**p<0.01, ***p<0.001, Student's t-test) C) TR activity measured using the SC-TR assay in 30 μg of protein from indicated cell lysates. The TR activity in LP9 mesothelial cells is 0.51 nmole/min or 17.U/mg. The TR activity in HM cells is 1.1 nmol/min or 35.6 U/mg. The TR activity in H2373 malignant mesothelioma cells is 1.14 nmol/min or 38 U/mg.

### DETAILED DESCRIPTION OF THE INVENTION

Thioredoxin reductase is an oxidoreductase which contributes to cellular redox homeostasis. This disclosure provides a method of determining the activity of TR in a biological sample. The assay provides an indication of TR activity by measuring the oxidation of NADPH. The assay is a direct assay because oxidation of NADPH (i.e., conversion of NADPH to NADP) is directly coupled to the conversion of TR from an oxidized state to a reduced state—which is necessary for its activity. As such, measurement of NADPH consumption is a direct measure of the TR activity. By measuring NADPH consumption continuously, a real-time measurement of TR activity can be obtained. Because the measurement of NADPH consumption can be carried out on a continuous basis, in specific embodiments, this assay is also a continuous and real-time assay.

In one aspect, the present disclosure provides a method for detecting and/or measuring TR activity in a test sample. The method comprises combining a biological sample from a subject—in which TR activity is to be determined—with NADPH and substrate for TR. In one embodiment, the substrate is a water-soluble diselenide containing molecule that can be reduced by TR. In one embodiment, the substrate is selenocystine. By following the conversion of NADPH to NADP, a direct indication of TR activity can be obtained. In one embodiment, the conversion of NADPH to NADP is obtained by measuring absorbance at 340 nm.

An advantage of the present assay is that non-ionic detergents do not interfere with the TR activity detection. For example, non-ionic detergents that can be used with this assay include NP-40 or Triton X-100. These buffers may contain protease inhibitors such as leupeptin and aprotinin as well as phosphatase inhibitors such as sodium orthovanadate. “Tris” based buffers (tris-hydroxymethyl aminomethane) can also be used with this assay.

The biological sample in which TR activity can be determined may be any biological sample comprising cells. For example, the sample may be a biological fluidic sample comprising cells such as blood, lymph etc., or may be a tissue sample, or a cell or tissue culture sample. Samples from individuals may be obtained by routine means including swabs, biopsies and the like.

In one embodiment, the subject is human being or a non-human animal. In one embodiment, the TR activity is determined in a sample comprising cultured or primary human or animal cells. Any organism that contains a high molecular weight. In general, low molecular weight (homodimers of about 35 kDa) thioredoxin reductases are found in prokaryotic bacteria, and the high molecular weight enzymes refer to homodimers with molecular weight of ˜110 kDa.

In one embodiment, the TR activity is determined by determining the conversion of NADPH to NADP as a function of time. For example, an assay for measurement of NADP/NADPH ratio may be used (by using commercially available kits). In one embodiment, the conversion can be determined by measuring the absorbance at about 340 nm (such as from 330 to 350 nm including all integers therebetween). In one embodiment, the absorbance is measured at 340 nm. As recited herein, and as will be understood by one skilled in the art, any reference to measurement of absorbance at 340 nm includes absorbance measurements around 340 (such as at any wavelength from 330 to 350 nm).

A convenient way of detecting the absorbance at 340 nm is to use a spectrophotometer, although any other means of detecting absorbance may also be used Any instrument that is equipped with a spectrophotometer such as a plate reader. An example of such is a BioTek Synergy HT. The conversion of NADPH to NADP is an indication of the NADPH consumption.

The present assay was observed to be specific. Thus, when TR activity was knocked down, such as by using siRNA or by using inhibitors of TR, very little change in absorbance at 340 could be detected. For an indication of the TR activity over time, while any two time points may be compared, in one embodiment, the starting point in an assay is when the sample, NADPH, and selenocystine are mixed, and this may be arbitrarily designated as time zero. Absorbance at 340 nm can then be monitored continuously from this point—or any arbitrarily selected point. In one embodiment, the background TR activity (e.g., NADPH conversion measured (when TR1 activity is knocked down by si RNA) as change in absorbance at 340 nm) over a period of up to 30 minutes is less than 10% compared to the sample in which TR1 activity was not knocked down by si-RNA. In various embodiments, the background TR activity is less than 9, 8, 7, 6, 5, 4, 3, 2, or 1%. In one embodiment, background activity may also be determined by eliminating one of the reaction components—such as selenocystine. Again, the background activity is less than 10% compared to the test reaction in which TR activity is being measure. This feature of low background provided an advantage over other TR assays—such as end-point assays (such as using insulin), which have exhibit higher background readings.

In one embodiment, the consumption of NADPH over time (such as by continuous measurement) in a test sample may be compared to that of a reference control thereby providing an indication of the TR activity in the test sample relative to the activity of the reference sample.

In another embodiment, the TR activity is determined by comparing the absorbance of a test sample at 340 nm at particular time point with that of a reference sample (control). The reference may be a positive or a negative control or both. A negative sample may be one in which the TR activity may be knocked down (by the use of inhibitors of the enzyme or by blocking the expression of the protein). A positive reference may be one which is known to have TR activity.

Although the present method can be used as an end-point assay, an advantage of the method of this disclosure is that TR activity can be determined on a continuous, real-time basis. This feature can be used to measure kinetics of inhibitors of TR. In one embodiment, the present disclosure provides a method for identifying inhibitors of TR and for measuring the kinetics of TR inhibitors, comprising the steps of contacting a reference sample comprising eukaryotic or bacterial cells with NADPH and selenocystine in the presence or absence of candidate inhibitors, and measuring the absorbance of 340 nm on a continuous basis or at desired intervals. Such intervals may range from 1 second to 1 minute or more. In one embodiment, the activity is measured under conditions when less than 5% of the substrate is consumed. In one embodiment, the activity was measured in the first 1, 2, 3, 4, or 5 minutes of the assay. Suitable inhibitors and the kinetics of inhibition can be determined from these measurements. In specific embodiments, the activity (absorbance at 340 nm) is measured for up to 30 minutes. For example, the activity may be measured for 5, 10, 15, 20, 25, and 30 minutes and all integers between 1 and 30 minutes. In one embodiment, the activity may be measured for less than one minute or longer than 30 minutes.

In one embodiment, the selenocystine is used in a pure form—as judged by a bright yellow color. If the selenocystine shows signs of impurity as evidenced by an orange color, this may be crystallized producing a relatively pure source of selenocystine.

In one aspect, the disclosure provides a kit for measurement of TR activity. The kit comprises NADPH, a diselenide substrate of TR (such as selenocystine), instructions for carrying out the assay, and optionally buffers. The buffers may be any of the buffers typically used in such reactions—including, but not limited to, phosphate buffers, Tris buffers, morpholine based buffers and the like. In one embodiment, the pH of the buffers is from 7.2 to 7.6 (including all values to the tenth decimal place therebetween). In one embodiment, it is 7.4. In one embodiment, the selenocystine is present in a pure form. In one embodiment, the buffers comprise non-ionic detergents.

The present disclosure is further illustrated by the following specific but non-limiting examples.

### EXAMPLE 1

### Materials and Methods

Materials. Selenocystine was purchased from Acros Organics (Morris Plains, N.J.). All other reagents were purchased from either Fisher Scientific (Fair Lawn, N.J.) or Sigma-Aldrich (St. Louis, Mo.) and were of reagent grade or better. CMRL and DMEM-F12 cell culture media was from Corning Cellgro (Manassas, Va.). Wild type TR1 plasmid (WT-TR1) was purchased from Ori-Gene (Rockville, Md., SKU: SC107562). TR1 primers were purchased from Integrated DNA Technologies (Coralville, Iowa). TR1 si-RNA was designed and purchased from Dharmacon (Pittsburgh, Pa.). Anti-TR1 antibody was purchased from Santa Cruz Biotechnology, Inc (Dallas, Tex.) and anti-Trx1 antibody was purchased from AbFrontier (Seoul, Korea). Anti-actin antibody, secondary antibodies, and Enhanced Chemiluminescent™ were purchased from Millipore (Billerica, Mass.). Auranofin was a gift from Pamela Cassidy of the University of Utah.

Preparation of selenocystine solution. L-selenocystine was purchased from Acros Organics as a technical grade yellow powder, and we recommend that the technical grade material be recrystallized. For recrystallization, the yellow crystals were dissolved in a minimal amount of 6 N HCl and then impurities removed with Whatman filter paper and a Buchner funnel. The filtrate was bright yellow, and next 10 N NaOH was added until yellow crystals appeared; the pH of the solution was then adjusted to 6-6.5 and the yellow crystals collected by filtration. The crystals were dried in an oven at 50° C. until the crystals did not clump together. Failure to dry the crystals results in an inaccurate gravimetric determination, resulting in inaccurate solution concentrations for the assay. Racemic selenocystine can also be used in the assay as TR reduces both L-selenocystine and racemic selenocystine with equal efficiencies. Because L-selenocystine has very limited solubility in water and does not dissolve easily in aqueous buffer at neutral pH, a 45.3 mM solution of L-selenocystine first was prepared by dissolving 15.3 mg of L-selenocystine in 200 μL of 1 N NaOH. To this solution was added 100 μL of 1 N HCl and then the solution was diluted to a 1 mL final volume with ddH₂O that is first filtered through 0.22 μm filters. The selenocystine was used at a final concentration of 800 μM in the assay.

Cell Culture. C10 mouse lung epithelial cells were maintained in CMRL cell culture media supplemented with 10% fetal bovine serum (FBS), 200 mM glutamine, and 0.5% penicillin streptomycin and propagated in a humidified incubator at 37° C. and 5% CO₂. Cells were trypsinized and re-plated to obtain 75% confluence on the following day for all subsequent experiments. Human malignant mesothelioma (HM, H2373) and immortalized but non-transformed mesothelial (LP9) cells were maintained in DMEM-F12 with hydrocortisone, insulin, transferrin, and selenite with 10% FBS.

Selenocystine TR assay. Cells were plated into 60 mm tissue culture dishes and the following day were washed with ice-cold 1× phosphate buffered saline (PBS) and lysed in the culture dish by adding either TE Buffer (50 mM Tris/HCl pH 7.5, 1 mM EDTA) or NP-40 lysis buffer (150 mM NaCl, 50 mM Tris pH 8.0, 1% NP-40, 1 μg/mL leupeptin, 1 μg/mL aprotinin, 1 mM NaF, 1 mM NaVO₃, 1 mM PMSF) as indicated in the text. Cell lysates were scraped from culture dishes on ice and transferred to 1.5 mL microcentrifuge tubes. TE buffer lysates were sonicated at 4° C. for 30 sec with 1-2 sec pulses using a sonic dismembrator. TE and NP-40 lysates were centrifuged for 10 min at 14,000 rpm at 4° C. and protein concentrations of the supernatants were determined by reading the absorption at 595 nm of Bradford reagent. A total reaction volume of 100 μl was assembled in a clear 96-well round bottom microplate containing the following: (i) master mix containing 1 mM NADPH and 2 mM selenocystine (SC) in ddH₂O (addition of 40 μl of master mix to 100 μl reaction volume yields final concentrations of 400 μM and 800 μM respectively), (ii) 25 μg or the largest practical amount of protein, (iii) TE or NP-40 lysis buffer. Controls for each sample included protein only control, protein and NADPH control, and protein and SC control. Control master mixes contained: (i) 2 mM NADPH in ddH₂O (addition of 20 μl to 100 μl reaction volume yields final concentration of 400 μM), (ii) 2 mM SC prepared as described above (addition of 40 μl to 100 μl reaction volume yields final concentration of 800 μM). An example of the typical reaction setup is given in Table 1.

TABLE 1
Volumes of reaction components for selenocystine assay.

| | Reaction components | | | |
|---|---|---|---|---|
| Sample | Experimental | Protein Control | NADPH Control | SC Control |
| 1 | 25.5 μL protein | 25.5 μL protein | 25.5 μL protein | 25.5 μL protein |
| | 34.5 μL lysis buffer | 74.5 μL lysis buffer | 54.5 μL lysis buffer | 34.5 μL lysis buffer |
| | 40 μL master mix | | 20 μL NADPH | 40 μl SC solution |
| 2 | 21.3 μL protein | 21.3 μL protein | 21.3 μL protein | 21.3 μL protein |
| | 38.7 μL lysis buffer | 78.7 μL lysis buffer | 58.7 μL lysis buffer | 38.7 μL lysis buffer |
| | 40 μL master mix | | 20 μL NADPH | 40 μL SC solution |
| 3 | 31 μL protein | 31 μL protein | 31 μL protein | 31 μL protein |
| | 29 μL lysis buffer | 69 μL lysis buffer | 49 μL lysis buffer | 29 μL lysis buffer |
| | 40 μL master mix | | 20 μL NADPH | 40 μL SC solution |

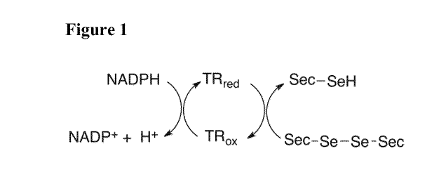

Each master mix was separately placed into a clean, sterile pipet basin and pipetted quickly into the wells of a 96-well microplate with a multichannel pipetter making sure to avoid bubbles. This approach was used in order that each sample was exposed to the master mix at the same time to ensure homogeneity of results. The plate was read in 30 sec intervals over a 20 min time period at 340 nm on a Synergy HT Microplate Reader™ (BioTek). Because the assay monitors the consumption of NADPH at 340 nm, an activity can be calculated by using Beer's Law and an extinction coefficient of 6,220 M⁻¹ cm⁻¹ for NADPH. The activity can be reported as mol NADPH consumed per min. Bradford assays for total protein concentration determination can be performed directly in each well of the 96-well plate. If this is done, then the activity can be reported as mol NADPH consumed per min per mg of total protein. For the data presented in FIGS. 2 and 5, 1 unit of TR activity is defined as the amount of TR that will consume 1 nmol of NADPH per min.

Discontinuous insulin end-point assay. Cell lysates were prepared in TE buffer as described above and the sonicated lysates were centrifuged for 10 min at 14,000 rpm at 4° C. Supernatants were transferred to fresh 1.5 mL microcentrifuge tubes and protein concentrations were determined by a Bradford assay. A total reaction volume of 80 μL was assembled by addition of 20 μL master mix to 20 μL of 25 μM E. coli Trx solution. The master mix contained 50 mM Tris/Cl pH 7.5, 1 mM EDTA, 900 μM NADPH (257 μM final), and 4.5 mg/mL insulin (1.3 mg/mL final). An example of the reaction setup for this end-point assay is given in Table 2. After addition of all of the components to a cuvette, the reactions were incubated at room temperature for 60 min and terminated by the addition of 930 μL of 6 M guanidine•HCL containing 1 mM 5,5′-dithio-bis(2-nitrobenzoic acid) (DTNB) solution prepared in ethanol; activity was detected by reading absorbance at 412 nm in a spectrophotometer. TR activity was determined by calculating the difference between identical samples +/− Trx. Here the activity is expressed as arbitrary units as determined by the raw A₄₁₂ reading. Alternatively, the number of units can be calculated by using Beer's Law and an extinction coefficient of 13,600 M⁻¹ cm⁻¹ for the TNB anion. One unit of TR activity is the amount of enzyme catalyzing the reduction of 1 μM of DTNB per min (formation of 2 μmol TNB anion).

TABLE 2
Insulin end-point assay reaction setup.

| | Lysis | | | Trx | Master |
|---|---|---|---|---|---|
| | buffer | Lysate | H₂O | stock | mix |
| Sample | (μL) | (μL) | (μL) | (μL) | (μL) |
| Blank + Trx | 40 | 0 | 0 | 20 | 20 |
| Blank − Trx | 40 | 0 | 20 | 0 | 20 |
| Sample #1 + Trx | 15 | 25 | 0 | 20 | 20 |
| Sample #1 − Trx | 15 | 25 | 20 | 0 | 20 |
| Sample #2 + Trx | 10 | 30 | 0 | 20 | 20 |
| Sample #2 − Trx | 10 | 30 | 20 | 0 | 20 |

TR over-expression and gene silencing. Full length TR1 was amplified from pCMV6-XL4 vector by PCR using specific forward (5′-GAAAGTCGAGGAGACAGTTAAGCATG-3′) and reverse (5′-CACAAGGAAAGGTCATGCTAAAACTG-3′) primers and subsequently cloned into pcDNA 3.1 mammalian expression vector. Insertion of WT-TR1 full-length cDNA, including the 3′ SECIS element, into pcDNA 3.1 was confirmed by sequencing using the appropriate forward and reverse primer sets (T7 Forward: BGH Reverse). WT-TR1 or empty pcDNA 3.1 vector were transfected into C10 cells according to manufacturer's protocol. Specific siRNA to TR1 (si-TR1, 5′-CCAUAGAGGGCGAAUUUAAUU-3′) and a control (“scramble”) RNA (5′-GCUCCUUUCGUCUCACAUAUU-3′) were introduced into C10 cells following the manufacturers guidelines (Dharmacon).

Immunoblotting. Equal protein amounts from cell lysates were separated on 10% gels by SDS-PAGE. Proteins were transferred to PVDF membrane for immunoblotting, and membranes then were blocked in 5% milk (Tris buffered saline with 0.1% Tween, TBST) for 1 hr at room temperature. Anti-TR1 (1:1000) anti-Trxl (1:2000) and anti-actin (1:5000) antibodies were diluted in 5% milk/TBST and incubated with membranes for 1 hr at room temperature. Membranes then were washed 5× with TBST and incubated with horseradish peroxidase (HRP) conjugated, secondary antibodies (1:5000) for 1 hr in 5% milk/TBST. Membranes were washed 5× with TBST and HRP secondary antibodies were detected using Enhanced Chemiluminescent™ solution and collected on X-ray film.

### Results

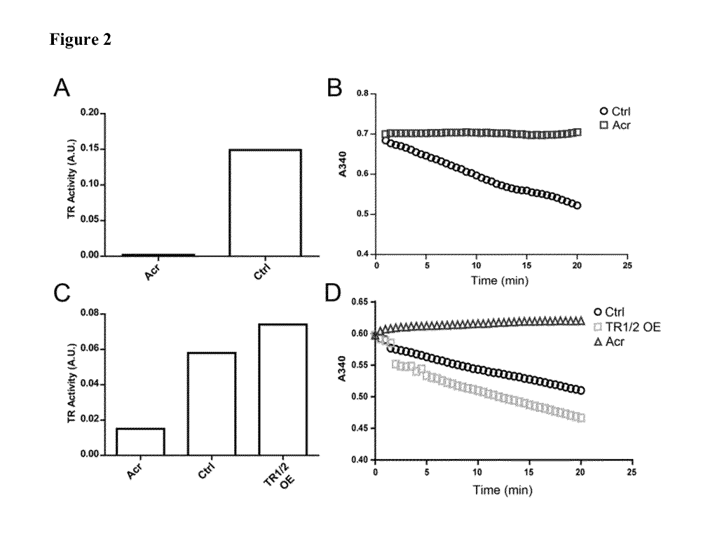

Utility of the assay using selenocystine as substrate for TR. To investigate using selenocystine as a substrate for determining thioredoxin reductase (TR) activity in cell lysates we compared the original insulin based method (referred to as end-point TR assay) to our alternative selenocystine TR assay (SC-TR assay). TR activity is determined in the SC-TR assay by monitoring the consumption of NADPH (340 nm) by TR in the reduction of selenocystine. FIG. 2A shows the inhibition of total TR activity from C10 cell lysates by the selenol-modifying reagent acrolein using the end-point TR assay. Acrolein directly alkylates nucleophiles, especially cellular thiols, of critical enzymes leading to a drastic reduction in their activity. Using the same cellular lysate from FIG. 2A, total TR activity was analyzed using the present assay. TR activity in untreated controls and complete inhibition of NADPH turnover in the presence of acrolein mimics the results of the end-point TR assay (FIG. 2B). These findings indicate that alkylation of TR and possibly other enzymes by acrolein inhibited TR activity as measured by both types of assays.

Specificity of SC-TR assay to thioredoxin reductase-1. Cytosolic TR1 and mitochondrial TR2 both contribute to maintaining a reduced thioredoxin pool, with TR1 responsible for the majority of TR activity in whole cell lysates. We therefore sought to determine the contribution of each type of TR to the reduction of selenocystine as measured in the SC-TR assay. C10 cells were transfected with TR1 and TR2 expression vectors and lysates were prepared in TE buffer to determine total TR activity 24 hrs after transfection (FIG. 2C and D). FIG. 2C shows an increase in total cellular TR activity using the end-point TR assay while FIG. 2D shows a similar increase in TR activity as determined by the SC-TR assay. In both assays over expression of TR1 and TR2 was inhibited to an equal extent by acrolein (FIGS. 2C and D). Overexpression of TR2 alone did not show an increase in TR activity by either assay (data not shown). Unless mitochondria are isolated free from cytosolic TR1, the modest contribution of mitochondrial TR2 to total TR activity could not be evaluated, further confirming that the contribution of TR2 activity is quite small in whole cell lysates. It is important to note that the buffers in commercially available mitochondrial isolation kits, which may contain reducing agents like DTT, do not interfere with the present assay.

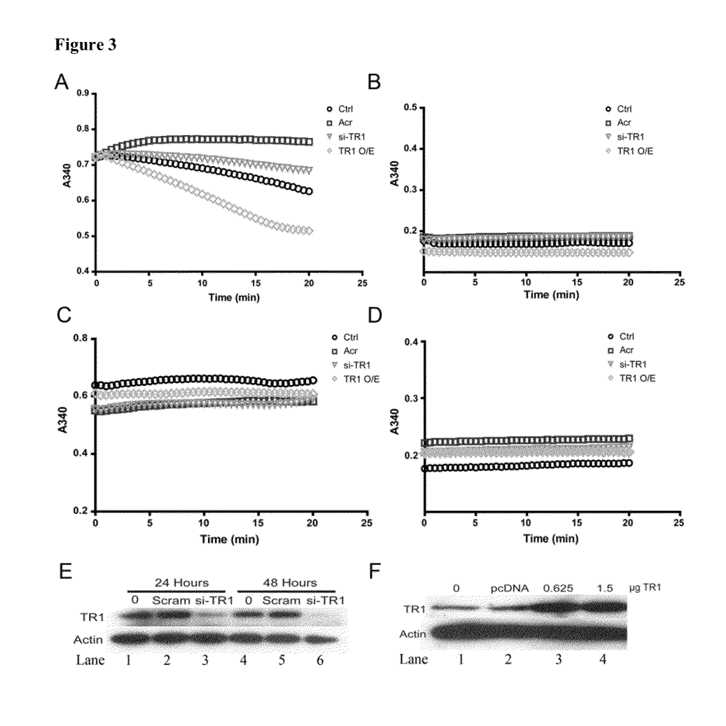

We further investigated the specificity of the SC-TR assay to cellular TR1 relative to the possible contributions made by other cellular enzymes by reducing the expression of TR1 protein using specific knockdown of TR1 by siRNA. Knockdown of TR1 by siRNA reduced the expression of TR1 for up to 48 hrs (FIG. 3E) and reduced TR activity below control levels as measured using the SC-TR assay (FIG. 3A). Conversely over-expression of cytosolic TR1 (FIG. 3F) increased total cellular TR activity significantly over control levels (FIG. 3A). To control for background activity as well as the contribution of the individual master mix constituents to the reaction, we also assayed protein only (FIG. 3B), master mix only (FIG. 3C) and NADPH only (FIG. 3D). These control reactions showed no difference in A₃₄₀ between the indicated experimental groups. Further, we confirmed that glutathione reductase, which reduces oxidized glutathione (GSSG) using NADPH, does not reduce selenocystine in vitro under the same experimental conditions that TR reduces selenocystine. These data indicate that the selenocystine-reductase activity measured in cell lysates is almost entirely due to TR activity and not other enzymatic activities. In addition, alterations in cellular TR protein expression levels correlate with selenocystine-reductase activity in cellular lysates.

Versatility of SC-TR assay in tissue lysis buffers. The constituents of buffers used in studying cellular signaling pathways often is a limiting factor in downstream assays that an investigator can employ using a single cell lysate. Accommodating different buffer components while obtaining optimal protein recovery for different purposes can be time consuming and technically difficult. Therefore we compared TR activity in samples prepared from whole cells lysed with buffers with or without the detergent NP-40 (a commonly used non-ionic detergent used in numerous cell lysis buffers) or TE buffer. Previous work in our lab indicated that non-ionic detergent based lysis buffers are not compatible with the end-point TR assay (such as the insulin assay).

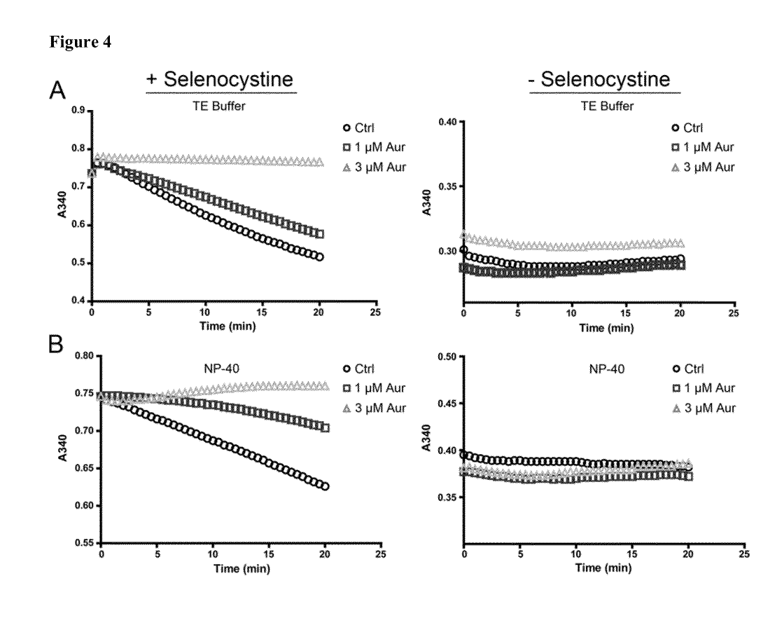

TR activity using the present assay was measured in lysates from cells treated with or without the specific TR inhibitor auranofin that were prepared with either TE buffer (FIG. 4A, left panel) or NP-40 buffer (FIG. 4B, left panel). TR activity using the SC-TR assays was nearly identical in either buffer, and the sensitivity to auranofin was also equivalent. To control for any background TR activity not due to the reduction of selenocystine in cell lysates, assays were performed in the presence of master mix (see Materials and Methods) without selenocystine (FIGS. 4A and 4B, right panels). This result shows that without addition of selenocystine to cell lysates, there is little or no endogenous activity that contributes to consumption of NADPH. The constituents of NP-40 lysis buffer, which also includes protease and phosphatase inhibitors, do not interfere with selenocystine-reductase activity in cell lysates, and suggest that this feature provides added flexibility for selecting lysis buffers that are compatible with a variety of downstream assays.

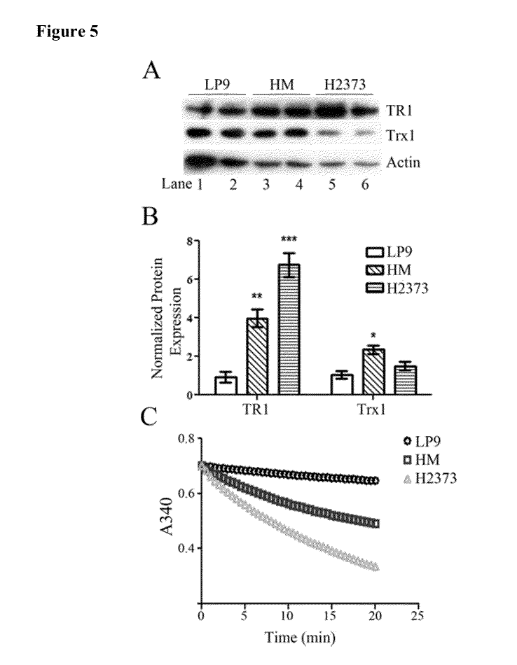

TR activity in human mesothelial and malignant mesothelioma cell lines. As noted in the introduction, TR expression levels vary in numerous disease states and may contribute to the progression of these diseases. Differential expression of antioxidant proteins and metabolic reprogramming has been observed in a number of cancers that positively influences proliferation and survival. The targeting of the altered redox status of cancerous tissues has been identified as a promising therapeutic target through pro-oxidant therapies that overwhelm the already increased tumor cell oxidative environment leading to cell death. We used our present assay to determine if TR activity is increased in malignant mesothelioma (MM) cells as compared to control LP9 mesothelial cells. This has previously been confirmed using the end-point assay (Newick et al., PLoS One. 7 (2012) e39404). Using the present assay, TR1 and Trx1 expression levels and total TR activity was measured in cell lysates prepared from MM cell lines (HM, H2373) and LP9 mesothelial cells. Immunoblots for TR1 expression showed MM cells have increased expression levels of TR1 as compared to LP9 cells (FIG. 5A). Trx1 is also overexpressed in MM cells, although not to the levels of TR1 (FIG. 5B). As a consequence of increased expression levels, TR activity is increased in MM cells compared to LP9 mesothelial control cells (FIG. 5C). Using our present assay we can determine that MM cell lines have twice the TR activity as compared to control LP9 cells as quantified by the present assay (35.6 U/mg and 38 U/mg for HM and H2373 cells, respectively, compared to 17.1 U/mg for LP9 cells). These findings support the use of the present assay for determining total TR activity from whole cell lysates.

The data presented herein describe a new assay for quantifying TR activity in whole cell lysates. The present assay uses selenocystine as a substrate for TR specific reduction, which is not a substrate for other NADPH cellular reductases. NADPH is consumed during the course of this reaction and can be monitored by spectrophotometry using either a cuvette, or in a multi-well plate reader. The multi-well plate format allows for the analysis of multiple experimental parameters at once, making the present amenable to experimental formats that require high throughput. Some distinct advantages of the present assay include: (i) compatibility with buffers that contain non-ionic detergents, (ii) being a continuous and direct assay that can be used in kinetic assays to calculate activity (iii) high specificity to TR, and (iv) being both less complex and less expensive to perform. Because the assay uses a direct substrate of TR, there is no need to supply exogenous Trx, insulin, and DTNB to the reaction mixture. The present assay can substitute for the previous end-point TR assay and may be advantageous in many contexts due to its adaptability to high throughput conditions.

The invention is not to be limited to the specific embodiments disclosed or the sample claims, and modifications and other embodiments are intended to be included within the scope of the disclosure.

1. A method of detecting thioredoxin reductase (TR) activity in a biological sample comprising combining the sample with NADPH and a water-soluble diselenide substrate of TR, and measuring conversion of NADPH to NADP over a period of time, wherein conversion of NADPH to NADP over time is an indication of the TR activity.

2. The method of claim 1, wherein the diselenide substrate of TR is selenocystine.

3. The method of claim 1, wherein the biological sample is a sample obtained from a subject.

4. The method of claim 3, wherein the subject is a human being.

5. The method of claim 4, wherein the biological sample is a biopsy sample from a tumor.

6. The method of claim 1, wherein the biological sample is a cell or tissue culture sample.

7. The method of claim 1, wherein the conversion of NADPH to NADP is measured continuously over a period of from 1 minute to 25 minutes.

8. The method of claim 1, wherein the method is carried out in the presence of non-ionic detergents.

9. A method of detecting thioredoxin reductase (TR) activity in a test biological sample comprising:
a) combining a water-soluble diselenide substrate of TR, NADPH, and the test biological sample; and
b) measuring conversion of NADPH to NADP;
c) comparing the conversion of NADPH to NADP in the test sample to that of a reference sample, wherein a difference in the conversion of NADPH to NADP between the test and the reference samples is an indication of the relative TR activity of the test sample compared to the reference sample.

10. The method of claim 9, wherein the reference sample is selected from the group consisting of a positive control or a negative control.

11. The method of claim 10, wherein the control sample is processed in parallel with the test sample.

12. The method of claim 9, wherein the test sample and the reference sample are from the same individual.

13. The method of claim 12, wherein the test sample is obtained from a tumor and the reference sample is obtained from normal tissue.

14. The method of claim 9, wherein the diselenide substrate of TR is selenocystine.

15. A kit for detection of TR activity comprising:
a) NADPH;
b) a water soluble diselenide substrate of TR; and
c) instructions for use of a) and b) in an assay for detection for TR.

16. The kit of claim 15, further comprising one or more buffers.
